(19) United States
(12) Patent Application Publication
(10) Pub. No.: US 2022/0233807 A1
(43) Pub. Date: Jul. 28, 2022

(54) MUSIC RECOMMENDATION FOR INFLUENCING PHYSIOLOGICAL STATE

(71) Applicant: Rubato, Inc., Pacific Palisades, CA (US)

(72) Inventors: Amit Sternberg, Pacific Palisades, CA (US); Noam Guy, Tel-Aviv (IL); Danny Portman, Johns Creek, GA (US)

(21) Appl. No.: 17/722,318

(22) Filed: Apr. 16, 2022

Related U.S. Application Data
(63) Continuation-in-part of application No. PCT/US20/56129, filed on Oct. 16, 2020.
(60) Provisional application No. 62/916,491, filed on Oct. 17, 2019.

(51) Int. Cl. A61M 21/02; G10H 1/00; G06F 3/16

(52) U.S. Cl. CPC A61M 21/02; G10H 1/0008; A61M 2021/0027; G10H 2250/311; G10H 2210/031; G06F 3/165

(57) ABSTRACT

Methods, systems, and devices are provided that allow for identifying the effects of musical selections on various biomarkers in one or more users. Based on the analysis of long- and short-term effects of musical attributes on biomarkers, music selections are recommended to achieve desired physiological effects and results.

### BACKGROUND

Music recommendation systems and services may aid a user discovering a selection of music. The recommendation may assist the user by narrowing the universe of all possible musical selections to a vastly reduced set that may be more easily traversed and more likely to be enjoyed by the user. A recommendation may be generated based on a variety of data, such as a user's profile or common characteristics of prior selections of music. Furthermore, a user may specify certain criteria, such as the musical genre or artist, to further refine the recommended selections.

### BRIEF DESCRIPTION OF THE DRAWINGS

The accompanying drawings, which are included to provide a further understanding of the disclosed subject matter, are incorporated in and constitute a part of this specification. The drawings also illustrate embodiments of the disclosed subject matter and together with the detailed description serve to explain the principles of embodiments of the disclosed subject matter. No attempt is made to show structural details in more detail than may be necessary for a fundamental understanding of the disclosed subject matter and various ways in which it may be practiced.

FIG. 1 shows a block diagram illustrating a process to recommend a selection of music according to an embodiment of the disclosed subject matter.

FIG. 2 shows a computing device according to an embodiment of the disclosed subject matter.

FIG. 3 shows a network configuration according to an embodiment of the disclosed subject matter.

FIG. 4 shows an example network and system configuration according to an embodiment of the disclosed subject matter

### DETAILED DESCRIPTION

The present subject matter may provide a musical recommendation to a user. Unlike conventional musical recommendation systems, the recommended musical selections may not necessarily be directed to those that the listener would likely purchase or even find enjoyable. Rather, the musical selection may be recommended based on its clinical effect on one or more measurable physiological states of the listener, known as a biomarker. A biomarker may be, for example, heart rate, heart rate variability, blood pressure, respiration rate, oxygen saturation, blood chemistry, perspiration, brain wave patterns, cardiac coherence, and the like.

The term “cardiac coherence” or “coherence” as used herein refers to a clinical indication that reflects the state of the autonomic nervous system and may be used to measure the balance ratio between the parasympathetic and sympathetic nervous systems. Cardiac coherence may be used to describe the measurement of the order, stability, and harmony in the oscillatory outputs of the bodily regulatory systems during a period of time.

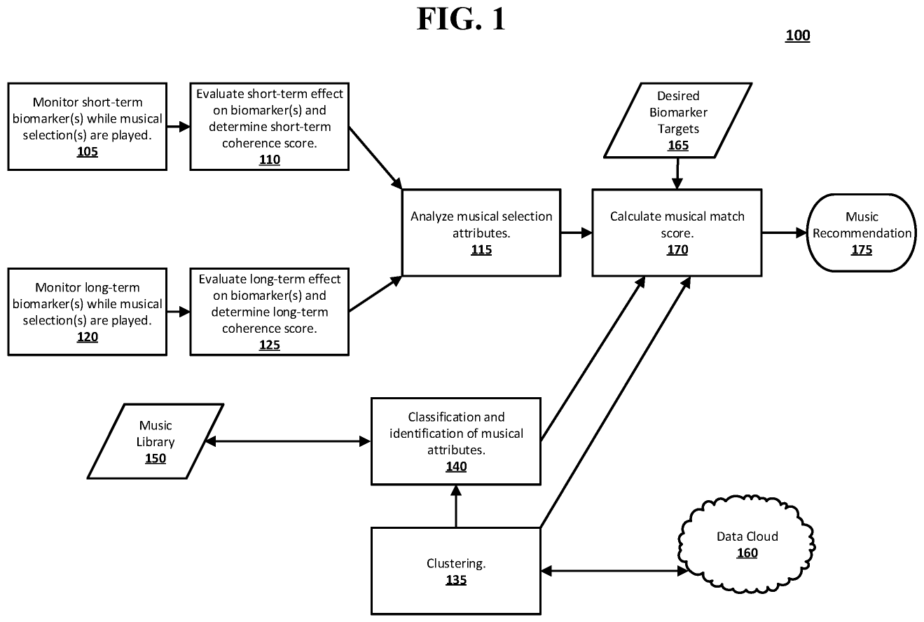

FIG. 1 illustrates a block diagram of a process 100 for recommending a musical selection in accordance with the present subject matter. Stages 105 and 110 may collectively represent an examination and analysis of the short-term clinical effects of a music selection on a user. A short-term period may be understood to mean within seconds, minutes, hours, or an interval having a duration of less than one day. In 105, one or more biomarkers of a user may be monitored while one or more selection of music are played. The selections of music may include one or more songs having a variety of musical attributes. The selections of music may be dynamically determined based on monitoring the user's biomarkers while the music is being played to attempt to identify the musical attributes having greater effect. The biomarkers may be monitored using suitable diagnostic sensors, devices, or through other testing methodologies. In an example, one biomarker may be the user's heart rate, which may be monitored using a smartwatch worn by the user. The short-term effect of playing the musical selection on the one or more biomarkers may be evaluated in 110. The evaluation may be based on comparing the one or more biomarkers monitored in 105 with one or more baseline biomarker measurements obtained when no musical selections are being played to assess the influence of a musical selection. The evaluation performed in 110 may produce a short-term coherence score based on a variety of criteria that reflects the short-term clinical impact on the autonomic nervous system of for each of the musical selections played during stage 105.

Stages 120 and 125 may collectively represent an examination and analysis of the long-term clinical effects of music on a user. A long-term period may be understood to mean an interval of one or more days, weeks, months, or years, but not less than one day. In 120, as previously described with respect to stage 105, one or more biomarkers of a user may be monitored while one or more selection of music are played. The selections of music may include one or more songs having a variety of musical attributes. The selections of music may be dynamically determined based on monitoring the user's biomarkers while the musical selection is being played to attempt to identify the musical attributes having greater influence or effect. The biomarkers may be monitored using suitable diagnostic sensors, devices, or through other testing methodologies. In an example, one biomarker may be the user's heart rate, which may be monitored using a smartwatch worn by the user. The long-term effect of playing the musical selection on the one or more biomarkers may be evaluated in 125. The evaluation may be based on comparing the one or more biomarkers monitored in 120 with one or more baseline biomarker measurements obtained when no musical selections are being played. The evaluation performed in 125 may produce a long-term coherence score based on a variety of criteria that reflects the long-term clinical impact on the autonomic nervous system for each of the musical selections played during stage 120.

In stage 115, the musical attributes of the selections played during 105 and/or 120 may be analyzed to identify which attributes are likely to have influenced the coherence score computed in 110. The analysis performed in 115 may consider, for example, the music's tempo or tempo range, the key, the tonality, the orchestration, the distribution of energy over a range of frequencies, and the like. Based on the short-term and long-term coherence scores generated in stages 110 and 125, as well as the musical selection attributes identified in 115, a musical match score may be generated in 170. The musical match score may include a variety of metrics that may quantify a correspondence between the musical selections played in stages 105 and 120, as well as the musical attributes associated with the musical selections, with one or more desired biomarker targets 165. A desired biomarker target 165 may be, for example, a heart rate that remains within a predetermined range. In an example, the musical match score may reveal that classical music selections having a tempo of about 120 beats-per-minute is more likely to influence a user's heart rate to remain between 50 and 60 beats per minute. For any set of desired biomarker targets 165, no single musical selection may be equally effective in causing the monitored biomarkers of the user to achieve those targets. In an embodiment, the desired biomarker targets 165 may be prioritized where the musical match score is computed to determine the musical selections that are more successful in causing the monitored biomarkers of the user to achieve the desired biomarker targets 165 having higher priority than other musical selections. A musical selection that is determined to be more likely to achieve the desired biomarker targets 165, based on the musical match score computed in 170, may be selected as the recommended music selection in 175.

Selections of music that are played in stages 105 and 120 may be sourced from a music library 150. Music library 150 may be implemented using a non-transitory computer-readable data store, such as database 15. Each musical selection stored within music library 150 may be periodically classified and processed in stage 140 to identify one or more musical attributes, such as tempo, range, key, tonality, orchestration, distribution of energy over a range of frequencies, and the like. The classification and processing may be carried out via a deep neural network (DNN), a recurrent neural network (RNN) or a long short-term memory network (LSTM), machine learning model, and the like. Data representative of the short-term and long-term effects of musical selections on various biomarkers across all participating users may be stored in a cloud-based data repository 160. The data stored in data repository 160 may be utilized during the musical match score computation in 170 to provide an additional basis for subsequently recommending a musical selection in 175. For example, stages 105, 110, 120, and 125 may be inconclusive and/or data obtained from a user may be sparse with respect to one or more biomarkers as to whether the determined musical selections would be effective in causing one or more desired biomarker targets 165 to be achieved. In this example, the musical match score 170 may leverage historical data collected from other users stored in data cloud 160 to “fill in the gaps” to guide the calculation based on what had been effective in other users or other similar users. Participating users for which data is stored in data cloud 160 may be clustered into similar groups in stage 135 such that the user being examined in stages 105, 110, 120, 125 may be compared with the clustered groups of users to aid in determining which musical selections may be more effective in causing the monitored biomarkers to achieve the desired biomarker targets 165. Other types of data stored in data cloud 160 may also be clustered, such as biomarkers determined to be influenced similarly by individual musical attributes. For example, clustering stage 135 may cluster heart rate and perspiration biomarkers in a same group determined to be similarly affected by musical selections having a relatively fast tempo.

In addition to the biomarker data collected in stages 105 and 120, additional user data may be used based on its availability to increase the granularity of the music recommendation generated in 175. For example, data representing the age, gender, weight, season, time of day in which a musical selection was played, location, weather, hours of sleep, and the like may be included within the musical match score computation 170 and the subsequent music recommendation made in 175. For example, based on performing evaluations 110, 125 over a long-term period with additional data indicating the number of hours that a user slept on a given day, process 100 may determine that recommending a musical selection having a relatively fast tempo may be more effective only where the user has slept more than four hours. In another example, process 100 may determine that the influence of a musical selection in reducing the heart rate of a user may vary depending on the current weather. In general, process 100 may attempt to utilize as much peripheral information as possible in generating the musical selection recommendation where the peripheral information may be deemed physiologically relevant and influential.

Desired biomarker targets 175 may be specified by a user collectively additionally and/or alternatively to being specified individually. In an embodiment, a user may specify the desired biomarker targets 175 by selecting a desired scenario. The scenario may include a predetermined selection of biomarker targets known through scientific research, experimental results stored in data cloud 160, user surveys, and the like, to stimulate the desired physiological outcome. In an example, a scenario may be “improve energy during aerobic workout,” which may include biomarker targets such as a increased intervals of high-frequency heart rate variability between 0.15 to 0.4 Hz and component and reduced intervals of low-frequency heart rate variability between 0.04 to 0.15 Hz. Other example scenarios may include, for example, “reduce stress,” “relax,” “prepare for public speaking,” “improve sex life,” and “improve sleep.” Selecting a scenario may collectively specify a set of desired biomarker targets 175 that may be utilized in computing the musical match score 170 and subsequently recommending a musical selection in 175.

As previously disclosed, “musical selections” as described herein may include one or more songs or similar discrete items. For example, embodiments disclosed herein may measure and/or predict beneficial effects of listening to one or more songs, such as to determine the effect of listening to a specific song or songs on a user's stress level based on one or more biomarkers. In addition to measuring and predicting the beneficial effects of listening to a specific song for the user's stress level or other impact on one or more biomarkers, embodiments disclosed herein may detect and/or measure the collective benefits of listening to a specific sequence of songs. A “sequence of songs” refers to a series of individual songs played in specific sequence, over the course of a short period of time, typically less than one hour. Accordingly, when a user has listened to two or more consecutive songs, the listening session is considered a “sequence of songs” or “song sequence”.

By measuring a biomarker such as the individual's stress level before and after listening to a song sequence, the physiological response of the user to the song sequence may be quantified separately from the physiological response of the user to any individual song in the sequence, or to a smaller sequence within the sequence. Embodiments disclosed herein then may aggregate the musical attributes of all the songs in the sequence to form a musical parameters representation of the entire song sequence, which is separate and distinct from the representation of any individual song in the sequence and may provide additional insight into the user's physiological response.

More specifically, any of the analyses previously disclosed herein may be performed with respect to a specific song sequence. For example, short- and/or long-term effect on one or more biomarkers, long- and/or short-term coherence scores, musical selection attributes, and musical match scores as previously disclosed with respect to FIG. 1 may be calculated with respect to a song sequence in the same manner as previously disclosed for one or more song selections. Song sequence scores may be determined by aggregating the values determined for individual songs, or by performing the same analysis with respect to the song sequence as a whole as opposed to individual songs.

It has been found that analyzing a song sequence in this manner provides additional benefit when compared to an analysis of the individual songs in the sequence, because it more closely resembles the way users tend to listen to music. For example, users often listen to a set series of songs in a playlist, a musical album or a other musical piece that is composed of many different parts that may be separated into multiple files. By applying the techniques disclosed herein to such a sequence, the associated recommendations and predictions provided by these embodiments often are more accurate than those that would be achieved by applying the same techniques to individual songs in the sequence. Furthermore, this approach allows the user's physiological response to be monitored and analyzed over a longer period of time, spanning multiple songs. This provides additional benefit over individual song analysis because it reduces and averages out point disturbances that may affect the physiological readings of a user before and after listening to just one song. For example, an individual song may have a beginning or ending portion that is musically distinct and different from the rest of the song and/or other songs in the sequence and thus may cause a temporary spike or drop in the user's physiological response, such as an apparent increase or decrease in heart rate or another biomarker. As another example, a longer work such as a musical album, soundtrack, preconstructed playlist or the like may have an overall physiological impact on the user which would be significantly less or would not be caused at all by a single song in the sequence. In an extreme example, a single song in a sequence may cause a temporary change in the user's physiological response different from the response caused by the entire sequence; in this case, considering the individual songs in the sequence may provide relatively inaccurate conclusions or predictions of the user's physiological response.

Thus, by treating song listening sessions as sequences of songs rather than isolated individual songs, and using the aggregated data (both physiological measurements and aggregated musical properties) across a sequence of songs, the predictive accuracy of the machine learning models disclosed herein is improved. It is believed that the overall higher predictive accuracy for sequences compared to individual songs primarily is due to data noise cancellation effects (as opposed to literal aural noise). For example, considering a user's stress trend over a longer period of time (e.g., 10-30 minutes as opposed to 3-6 minutes), the impact of listening to music is more observable. If the majority of songs in a sequence have a favorable effect, the sequence will also have a favorable stress reduction effect. Further, detrimental or non-beneficial effects of a single song may be reduced, eliminated, or even reversed into beneficial effects based on the song's relationship to others in the sequence as perceived by the user.

Notably, embodiments disclosed herein may provide various benefits to the computer systems on which they are implemented. For example, as music recommendation systems such as automated playlists, music discovery services and systems, and the like become more popular, there is an increased need for the recommendation to be more accurate in identifying recommendations that are suitable for individual users. Conventional recommendation systems typically rely on similarities between individual songs or other musical works without regard to the effect of those songs on the user listener. By incorporating the techniques and systems disclosed herein, the accuracy and usefulness of music recommendation systems may be improved on a user-by-user basis. Furthermore, the efficiency of the computerized music recommendation system itself may be improved. Conventional systems may require much larger libraries of prior “good” recommendations (as indicated by user acceptance) to provide new equally “good” recommendations to a new user, thus requiring large amounts of storage, on-demand processing (such as to evaluate a new library of music), or other computing resources. Techniques disclosed herein allow for much faster tailoring of recommendations to individual users, which may be further tailored based on the user's physiological response and/or the user's desired physiological effect. This requires less historical data and fewer prior “good” recommendations to achieve an acceptably-accurate recommendation for any individual user, thereby reducing the computing resources required to do so.

In situations in which the systems discussed here collect personal information about users, or may make use of personal information, the users may be provided with an opportunity to control whether programs or features collect user information (e.g., information about a user's social network, social actions or activities, profession, a user's preferences, or a user's current location), or to control whether and/or how to receive content from the content server that may be more relevant to the user. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over how information is collected about the user, which data is stored in data cloud 160, and how that data is used by a system as disclosed herein.

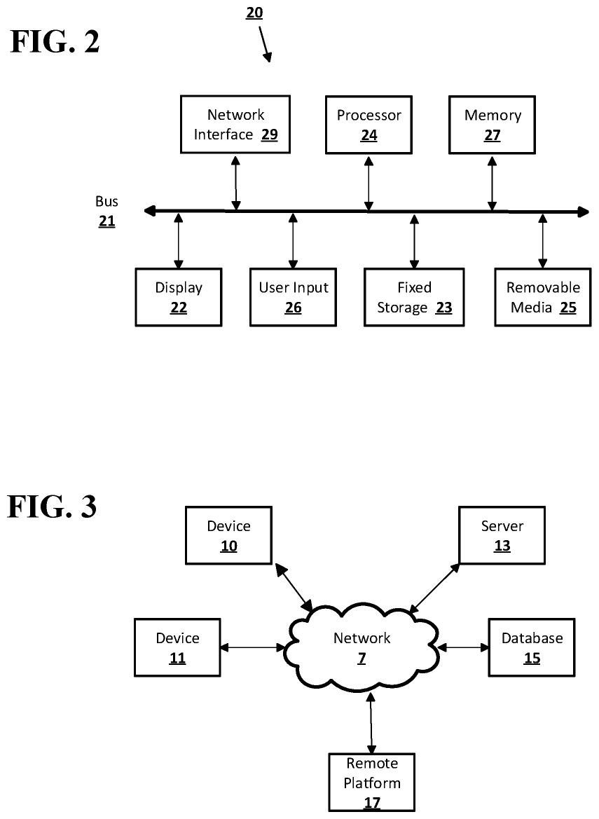

Embodiments of the presently disclosed subject matter may be implemented in and used with a variety of component and network architectures. FIG. 2 is an example computing device 20 suitable for implementing embodiments of the presently disclosed subject matter. The device 20 may be, for example, a desktop or laptop computer, or a mobile computing device such as a smart phone, tablet, or the like. The device 20 may include a bus 21 which interconnects major components of the computer 20, such as a central processor 24, a memory 27 such as Random Access Memory (RAM), Read Only Memory (ROM), flash RAM, or the like, a user display 22 such as a display screen, a user input interface 26, which may include one or more controllers and associated user input devices such as a keyboard, mouse, touch screen, and the like, a fixed storage 23 such as a hard drive, flash storage, and the like, a removable media component 25 operative to control and receive an optical disk, flash drive, and the like, and a network interface 29 operable to communicate with one or more remote devices via a suitable network connection.

The bus 21 allows data communication between the central processor 24 and one or more memory components, which may include RAM, ROM, and other memory, as previously noted. Typically RAM is the main memory into which an operating system and application programs are loaded. A ROM or flash memory component can contain, among other code, the Basic Input-Output system (BIOS) which controls basic hardware operation such as the interaction with peripheral components. Applications resident with the computer 20 are generally stored on and accessed via a computer readable medium, such as a hard disk drive (e.g., fixed storage 23), an optical drive, floppy disk, or other storage medium.

The fixed storage 23 may be integral with the computer 20 or may be separate and accessed through other interfaces. The network interface 29 may provide a direct connection to a remote server via a wired or wireless connection. The network interface 29 may provide such connection using any suitable technique and protocol as will be readily understood by one of skill in the art, including digital cellular telephone, WiFi, Bluetooth®, near-field, and the like. For example, the network interface 29 may allow the computer to communicate with other computers via one or more local, wide-area, or other communication networks, as described in further detail below.

Many other devices or components (not shown) may be connected in a similar manner (e.g., document scanners, digital cameras and so on). Conversely, all of the components shown in FIG. 2 need not be present to practice the present disclosure. The components can be interconnected in different ways from that shown. The operation of a computer such as that shown in FIG. 2 is readily known in the art and is not discussed in detail in this application. Code to implement the present disclosure can be stored in computer-readable storage media such as one or more of the memory 27, fixed storage 23, removable media 25, or on a remote storage location.

FIG. 3 shows an example network arrangement according to an embodiment of the disclosed subject matter. One or more devices 10, 11, such as local computers, smart phones, tablet computing devices, and the like may connect to other devices via one or more networks 7. Each device may be a computing device as previously described. The network may be a local network, wide-area network, the Internet, or any other suitable communication network or networks, and may be implemented on any suitable platform including wired and/or wireless networks. The devices may communicate with one or more remote devices, such as servers 13 and/or databases 15. The remote devices may be directly accessible by the devices 10, 11, or one or more other devices may provide intermediary access such as where a server 13 provides access to resources stored in a database 15. The devices 10, 11 also may access remote platforms 17 or services provided by remote platforms 17 such as cloud computing arrangements and services. The remote platform 17 may include one or more servers 13 and/or databases 15.

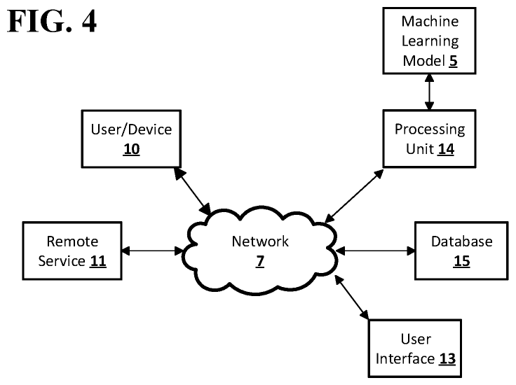

FIG. 4 shows an example arrangement according to an embodiment of the disclosed subject matter. One or more devices or systems 10, 11, such as remote services or service providers 11, user devices 10 such as local computers, smart phones, tablet computing devices, and the like, may connect to other devices via one or more networks 7. The network may be a local network, wide-area network, the Internet, or any other suitable communication network or networks, and may be implemented on any suitable platform including wired and/or wireless networks. The devices 10, 11 may communicate with one or more remote computer systems, such as processing units 14, databases 15, and user interface systems 13. In some cases, the devices 10, 11 may communicate with a user-facing interface system 13, which may provide access to one or more other systems such as a database 15, a processing unit 14, or the like. For example, the user interface 13 may be a user-accessible web page that provides data from one or more other computer systems. The user interface 13 may provide different interfaces to different clients, such as where a human-readable web page is provided to a web browser client on a user device 10, and a computer-readable API or other interface is provided to a remote service client 11.

The user interface 13, database 15, and/or processing units 14 may be part of an integral system or may include multiple computer systems communicating via a private network, the Internet, or any other suitable network. One or more processing units 14 may be, for example, part of a distributed system such as a cloud-based computing system, search engine, content delivery system, or the like, which may also include or communicate with a database 15 and/or user interface 13. In some arrangements, a machine learning model 5 may provide back-end processing, such as where stored or acquired data is pre-processed by the machine learning model 5 before delivery to the processing unit 14, database 15, and/or user interface 13. For example, a machine learning model 5 may provide various prediction models, data analysis, or the like to one or more other systems 13, 14, 15.

More generally, various embodiments of the presently disclosed subject matter may include or be embodied in the form of computer-implemented processes and apparatuses for practicing those processes. Embodiments also may be embodied in the form of a computer program product having computer program code containing instructions embodied in non-transitory and/or tangible media, such as floppy diskettes, CD-ROMs, hard drives, USB (universal serial bus) drives, or any other machine readable storage medium, such that when the computer program code is loaded into and executed by a computer, the computer becomes an apparatus for practicing embodiments of the disclosed subject matter. Embodiments also may be embodied in the form of computer program code, for example, whether stored in a storage medium, loaded into and/or executed by a computer, or transmitted over some transmission medium, such as over electrical wiring or cabling, through fiber optics, or via electromagnetic radiation, such that when the computer program code is loaded into and executed by a computer, the computer becomes an apparatus for practicing embodiments of the disclosed subject matter. When implemented on a general-purpose microprocessor, the computer program code segments configure the microprocessor to create specific logic circuits.

In some configurations, a set of computer-readable instructions stored on a computer-readable storage medium may be implemented by a general-purpose processor, which may transform the general-purpose processor or a device containing the general-purpose processor into a special-purpose device configured to implement or carry out the instructions. Embodiments may be implemented using hardware that may include a processor, such as a general purpose microprocessor and/or an Application Specific Integrated Circuit (ASIC) that embodies all or part of the techniques according to embodiments of the disclosed subject matter in hardware and/or firmware. The processor may be coupled to memory, such as RAM, ROM, flash memory, a hard disk or any other device capable of storing electronic information. The memory may store instructions adapted to be executed by the processor to perform the techniques according to embodiments of the disclosed subject matter.

The foregoing description, for purpose of explanation, has been described with reference to specific embodiments. However, the illustrative discussions above are not intended to be exhaustive or to limit embodiments of the disclosed subject matter to the precise forms disclosed. Many modifications and variations are possible in view of the above teachings. The embodiments were chosen and described in order to explain the principles of embodiments of the disclosed subject matter and their practical applications, to thereby enable others skilled in the art to utilize those embodiments as well as various embodiments with various modifications as may be suited to the particular use contemplated.

What is claimed is:

1. A method comprising:
monitoring a biomarker of a user while a first music item is played for the user, the first music item having a plurality of musical attributes;
determining a short-term effect of the musical selection on the biomarker;
identifying a first musical attribute of the plurality of musical attributes that is likely to have influenced the short-term effect of the musical selection; and
based upon the first musical attribute, generating a musical match score that identifies a correspondence between the first musical attribute and a biomarker target;
based upon the musical match score and the biomarker target, identifying a recommended music selection; and
providing the recommended music selection to the user.

2. The method of claim 1, further comprising:
determining a long-term effect of the musical selection on the biomarker; and
identifying a second musical attribute of the plurality of musical attributes that is likely to have influenced the long-term effect of the musical selection;
wherein the musical match score is further based on the second musical attribute and identifies a correspondence between the second musical attribute and the biomarker target.

3. The method of claim 2, wherein the step of determining the long-term effect of the musical selection on the biomarker comprises comparing a measurement of the biomarker to a baseline measurement of the biomarker when no musical selection is played for the user.

4. The method of claim 2, further comprising generating a long-term coherence score that indicates the long-term clinical impact of the musical item on the autonomic nervous system of the user.

5. The method of claim 1, wherein the step of determining the short-term effect of the musical selection on the biomarker comprises comparing a measurement of the biomarker to a baseline measurement of the biomarker when no musical selection is played for the user.

6. The method of any claim 1, further comprising generating a short-term coherence score that indicates the short-term clinical impact of the musical item on the autonomic nervous system of the user.

7. The method of claim 1, wherein the first music item comprises a sequence of songs or other musical selections.

8. The method of claim 7, wherein the sequence of songs or other musical selections comprises songs selected from a digital music library.

9. The method of claim 8, further comprising periodically classifying musical selections in the digital music library to classify each musical selection in the library based upon one or more musical attributes of the selection.

10. The method of claim 9, wherein the classification is performed by a deep neural network, a recurrent neural network, a long short-term memory network, or a combination thereof.

11. The method of claim 1, wherein the musical match score is based upon a plurality of musical attributes of the first music item.

12. The method of claim 1, wherein the step of monitoring the biomarker comprises monitoring a plurality of biomarkers and determining a short-term effect on each biomarker of the plurality of biomarkers.

13. The method of claim 1, wherein the biomarker comprises a biomarker selected from the group consisting of the user's heart rate, heart rate variability, blood pressure, respiration rate, oxygen saturation, blood chemistry, perspiration, brain wave patterns, cardiac coherence, or a combination thereof.

14. The method of claim 1, wherein the biomarker is monitored using a phone and/or a smart watch worn by the user.

15. The method of claim 1, further comprising:
collecting historical data from a plurality of users, the historical data indicating the short-term and/or long-term effect of the first music item on the first biomarker for the respective user;
wherein the musical match score is further based upon the historical data.

16. The method of claim 15, further comprising:
clustering the historical data based upon biomarkers influenced similarly by individual musical attributes.

17. The method of claim 1, wherein the biomarker target is specified by the user.

18. The method of claim 17, wherein the biomarker target is specified by the user via a selection of a specified physiological outcome.

19. A device comprising:
a processor; and
a computer-readable medium storing a plurality of instructions which cause the processor to:
monitor a biomarker of a user while a first music item is played for the user, the first music item having a plurality of musical attributes;
determine a short-term effect of the musical selection on the biomarker;
identify a first musical attribute of the plurality of musical attributes that is likely to have influenced the short-term effect of the musical selection; and
based upon the first musical attribute, generate a musical match score that identifies a correspondence between the first musical attribute and a biomarker target;
based upon the musical match score and the biomarker target, identify a recommended music selection; and
provide the recommended music selection to the user

20. A non-transitory computer-readable medium storing executable instructions which, when executed by a computer processor, causes the processor to perform a method comprising:
monitoring a biomarker of a user while a first music item is played for the user, the first music item having a plurality of musical attributes;
determining a short-term effect of the musical selection on the biomarker;
identifying a first musical attribute of the plurality of musical attributes that is likely to have influenced the short-term effect of the musical selection; and
based upon the first musical attribute, generating a musical match score that identifies a correspondence between the first musical attribute and a biomarker target;
based upon the musical match score and the biomarker target, identifying a recommended music selection; and
providing the recommended music selection to the user.
